(19) United States
(12) Patent Application Publication
(10) Pub. No.: US 2026/0232466 A1
(43) Pub. Date: Aug. 13, 2026

(54) STENT GRAFTS AND RELATED DELIVERY SYSTEMS

(71) Applicant: Mayo Foundation for Medical Education and Research, Rochester, MN (US)

(72) Inventor: Tiziano Tallarita, Altoona, WI (US)

(21) Appl. No.: 19/472,899

(22) PCT Filed: Apr. 8, 2024
(86) PCT No.: PCT/US2024/023577

§ 371 Date: Oct. 6, 2025

Related U.S. Application Data
(60) Provisional application No. 63/457,609, filed on Apr. 6, 2023.

(51) Int. Cl. A61F 2/95; A61F 2/966

(52) U.S. Cl. CPC A61F 2/9517; A61F 2/966

(57) ABSTRACT

A stent graft includes a stent having a proximal end and a distal end and a tubular body disposed longitudinally there-between. The stent includes a plurality of struts forming the tubular body. The stent graft includes a stent graft material coupled to a portion of the tubular body. The stent graft material defines an opening. The stent graft further includes one or more rings engaged with an edge of the stent, the one or more rings configured to receive a string, wherein tensioning of the string causes the stent graft to be in a radially collapsed configuration.

### CROSS-REFERENCE TO RELATED APPLICATIONS

This application claims the benefit of U.S. Provisional Application Ser. No. 63/457,609, filed Apr. 6, 2023. The disclosure of the prior application is considered part of the disclosure of this application, and is incorporated in its entirety into this application.

### TECHNICAL FIELD

This disclosure describes stent grafts and stent graft delivery systems configured to deploy the stent grafts within a blood vessel of a subject in need thereof.

### BACKGROUND

Endovascular repair of complex aortic aneurysms (EVAR) can be done using fenestrated or branched devices (F-BEVAR) or parallel stent grafts. These techniques require significant device and catheter manipulations that can result in embolic events and vessel injury. Microembolization of cholesterol fragments or macroscopic particles of thrombus and plaque can result in end-organ damage and loss of renal function, bowel infarction, spinal cord injury (SCI), or stroke. The presence of aortic thrombus has been associated with mesenteric and renal ischemia in patients treated by fenestrated endografts. Irregular thrombus or debris in the thoracic and abdominal aorta has been identified as a predictor of renal deterioration. A study found aortic thrombus was present in 95% of patients treated with fenestrated-branched stent graft, with more than 50% of these thrombi being pedunculated and at high risk for embolization to the visceral organs.

Furthermore, a thrombus in the visceral segment of the aorta or in proximity of one or more vital side branches is not feasible with the currently available aortic stents. In the presence of pedunculated thrombus in the visceral segment of the aorta, many providers deem the patients unsuitable for an endovascular repair. Some providers with high-risk patients and no open surgical option, decide together with the patients to accept the risk of embolization and perform the endovascular procedure anyway.

As such, there is a clear need for a solution for the treatment of complex aortic aneurysms, including those found in the thoracic and abdominal (e.g., visceral segment) of the aorta.

### SUMMARY

In general, this disclosure describes stent grafts and stent graft delivery systems. Such stent grafts can be used for the treatment of an aneurysm and are configured to be deployed within a blood vessel of a patient.

In one aspect, this disclosure is directed to a stent graft including: a stent having a proximal end and a distal end and a tubular body disposed longitudinally therebetween, the stent comprising a plurality of struts forming the tubular body; a stent graft material coupled to a portion of the tubular body, the stent graft material defining an opening; and one or more rings engaged with an edge of the stent, the one or more rings configured to receive a string, wherein tensioning of the string causes the stent graft to be in a radially collapsed configuration.

Embodiments may include one or more of the following features.

In some embodiments, the stent graft material is coupled to about 40% to about 60% of the tubular body.

In some embodiments, the stent comprises a first label, and the stent graft material comprises a second label such that the first and second labels are configured to align when the stent graft is in a deployed state.

In some embodiments, the opening defined by the stent graft material is configured to be aligned with a blood vessel opening.

In some embodiments, the tubular body is a metal structure, and wherein the stent graft material is an impermeable fabric.

In some embodiments, loosening of the string causes the stent graft to be in a radially expanded configuration.

In some embodiments, the plurality of struts are interconnected.

In some embodiments, the plurality of struts are arranged in a diamond-shaped pattern.

In some embodiments, the plurality of struts is a first plurality of struts, and the stent further comprises a second plurality of struts and a third plurality of struts arranged in a diamond-shaped pattern, and wherein the stent graft further comprises one or more rings engaged at one or more apexes of one or more diamonds forming the diamond-shaped pattern.

In another aspect, this disclosure is directed to a stent graft delivery system including: an elongated shaft having a proximal end and a distal end; a stent graft having a tubular body disposed over the elongated shaft, the stent graft comprising a plurality of struts forming the tubular body and one or more rings engaged with an edge of the stent, the one or more rings configured to receive a string; and a handle assembly extending from a proximal end of the elongated shaft, the handle assembly attached to the string and configured to tension or loosen the string, wherein the stent graft is configured to be in a radially expandable configuration when the string is loosened, and wherein the stent graft is configured to be in a radially collapsed configuration when the string is tensioned.

In some embodiments, the stent graft is configured to reversibly transition between a radially expandable configuration and the radially collapsed configuration.

In some embodiments, the handle assembly further comprises a blade configured to sever the string.

In some embodiments, the stent graft is configured to be deployed and engaged with a wall of a blood vessel after the string is severed.

In some embodiments, the handle assembly further comprises a safety pin configured to prevent the blade from contacting the string.

In some embodiments, the stent graft delivery system further includes an elongated, tubular sheath configured to be disposed over the stent graft when the stent graft is in the radially collapsed configuration.

In some embodiments, the elongated shaft is hollow and defines a lumen, and wherein the handle assembly further comprises a side port in fluid communication with the lumen of the elongated shaft.

In some embodiments, the plurality of struts are reversibly collapsible.

In some embodiments, the plurality of struts are arranged in a diamond-shaped pattern.

In some embodiments, the plurality of struts is a first plurality of struts, and the stent graft further comprises a second plurality of struts and a third plurality of struts arranged in a diamond-shaped pattern, and wherein the stent graft further comprises one or more rings engaged at one or more apexes of one or more diamonds forming the diamond-shaped pattern.

In some embodiments, the string is a first string, wherein the one or more rings of the second plurality of struts is configured to receive a second string, wherein the one or more rings of the third plurality of struts is configured to receive a third string, and wherein the handle assembly is attached to the second and third strings.

Some embodiments of the stent grafts and stent graft delivery systems described herein may provide one or more of the following advantages. First, some embodiments described herein provide a stent graft that can be reversibly positioned within a blood vessel. For example, in some embodiments, the stent grafts of the disclosure may allow the user (e.g., a medical practitioner such as a surgeon) to reposition the stent graft as needed in a quick and easy manner (e.g., by only sliding a handle in a handle assembly in order to collapse or expand the stent graft and recapture the stent graft with a sheath).

Second, some embodiments described herein provide a stent graft that is configured to have its proximal, middle, and distal regions controlled and manipulated (e.g., reversibly transitioned between a radially collapsed configuration and a radially expanded configuration). This design may advantageously allow the user to reversibly and individually adjust each region of the stent graft as needed, which further enables the user to achieve better positioning and engagement of the stent graft with the blood vessel wall.

Third, some embodiments described herein provide a stent graft that can accommodate one or more openings of one or more blood vessels that branch off of a main blood vessel (e.g., an abdominal aorta and/or a thoracic aorta) in order to preserve these vital, branching blood vessels. Furthermore, the size of the opening of the stent graft as well as the diameter and length of the stent graft can be adjusted based on the anatomy of an individual patient, which can be determined by conventional imaging techniques (e.g., magnetic resonance imaging (MRI) and/or computerized tomography (CT)).

Fourth, some embodiments described herein provide a stent graft that has side holes in the distal part of the supporting catheter to allow obtaining angiograms without the need of creating a second vascular access (e.g., via a contralateral groin) and using an additional catheter (e.g., a flushing catheter), as described in more detail below.

The use of the term “about,” as used herein, refers to an amount that is near the stated amount by about 10% including increments therein. For example, “about” can mean a range including the particular value and ranging from 10% below that particular value and spanning to 10% above that particular value.

Unless otherwise defined, all technical and scientific terms used herein have the same meaning as commonly understood by one of ordinary skill in the art to which this disclosure pertains. Although methods and materials similar or equivalent to those described herein can be used to practice the various embodiments, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the disclosure are set forth in the accompanying drawings and the description below. Other features, objects, and advantages of the methods and devices described will be apparent from the description, the drawings, and the claims.

### DESCRIPTION OF DRAWINGS

FIG. 1A is a front view of a stent graft in a flat configuration.

FIG. 1B is a perspective view of the stent graft of FIG. 1A in a cylindrical configuration.

FIG. 2A is a partial perspective view of a stent graft delivery system.

FIG. 2B is a partial perspective view of a stent graft of the stent graft delivery system of FIG. 2A.

FIGS. 3A-3D are partial perspective views of a handle assembly of the stent graft delivery system of FIG. 2A.

FIGS. 4A-4E are partial perspective views of the stent graft delivery system of FIG. 2A at radially expanded, transitory, and collapsed configurations.

FIGS. 5A-5D are partial perspective views of the stent graft delivery system of FIG. 2A illustrating the engagement and disengagement of a safety pin.

FIG. 6 is a perspective view of an exemplary stent graft delivery system.

FIG. 7 is an enlarged, perspective view of the rings of the stent graft delivery system of FIG. 6.

FIG. 8 is an enlarged, perspective view of the rings of the handle assembly of FIG. 6.

FIGS. 9A-9D are perspective views of a stent graft of the stent graft delivery system of FIG. 6.

FIGS. 10A-10C are partial perspective views of a stent of the stent graft delivery system of FIG. 6.

FIG. 11 is a perspective view of an exemplary stent graft delivery system.

FIGS. 12A and 12B are partial perspective views of a stent graft of the stent graft delivery system of FIG. 11.

FIG. 13 is a partial perspective view of a handle assembly of the stent graft delivery system of FIG. 11.

FIG. 14 is a partial enlarged view of the handle assembly of the stent graft delivery system of FIG. 11.

Like reference numbers represent corresponding parts throughout.

### DETAILED DESCRIPTION

This document relates to stent grafts and stent graft delivery systems for the deployment of stent grafts within a blood vessel of a subject in need thereof. In some embodiments, the blood vessel is the aorta. In some embodiments, the blood vessel is a thoracic aorta, an abdominal aorta, or both. In some embodiments, the stent grafts of the disclosure define an opening that is aligned with one or more branching blood vessels in order to maintain the openings of these branching blood vessels open to ensure blood flow through these openings is preserved. In some embodiments, non-limiting examples of the branching blood vessel include an intercostal artery, a superior mesenteric artery, an inferior mesenteric artery, a subclavian artery, a renal artery, a gonadal artery, a celiac trunk, a common iliac artery, a left common carotid artery, and a brachiocephalic artery. In some embodiments, the stent grafts of the disclosure are used to treat penetrating aortic ulcers or thrombus where there is the need to preserve vital arterial branches, an aneurysm (e.g., a saccular aneurysm or a complex aneurysm) where there is the need to preserve vital arterial branches, and/or to tackle dissection flaps where there is the need to preserve vital arterial branches.

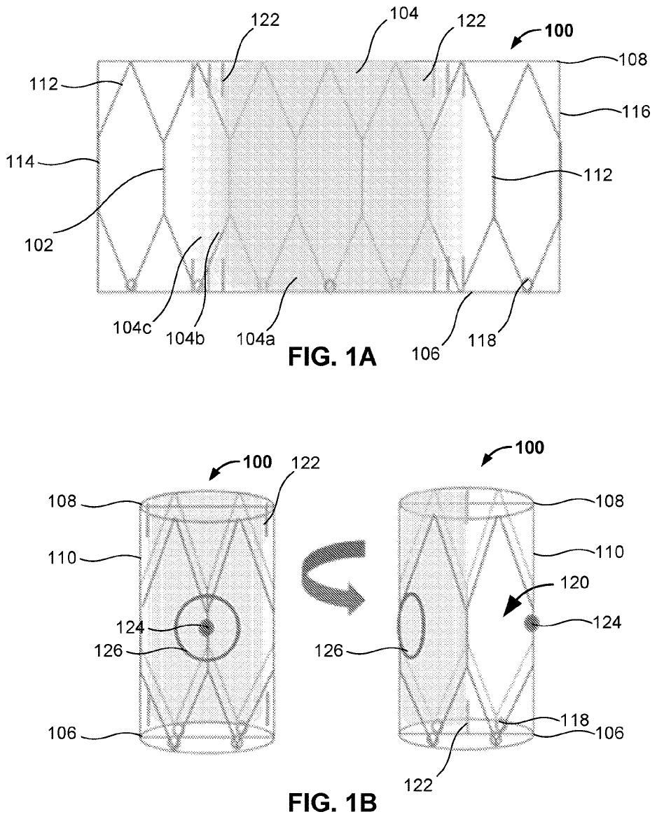

FIGS. 1A and 1B illustrate a stent graft 100 that is configured to be deployed within a lumen of a blood vessel in a subject (e.g., a patient) in need thereof. The stent graft 100 can be used to treat an aneurysm of the subject. For example, in some embodiments, the stent graft 100 can be used to treat complex aneurysms such as, but not limited to, thoracic aortic aneurysms, abdominal aortic aneurysms (e.g., juxtarenal abdominal aortic aneurysms), and thoracic aortic aneurysms. The stent graft 100 includes a stent 102, a stent graft material 104, and one or more rings 118.

Referring specifically to FIG. 1A, the stent 102 has a rectangular shape when in a flat configuration. The stent 102 has a proximal end 106 and a distal end 108 and opposing first and second lateral edges 114 and 116. The stent 102 has a tubular body 110 disposed longitudinally between the proximal and distal ends 106, 108 when in a tubular configuration, as shown in FIG. 1B. The stent 102 includes a plurality of struts 112 that form the tubular body 110 and provide structural support to the stent graft 100. The plurality of struts 112 are interconnected and arranged in a diamond-like pattern. For example, each strut of the plurality of struts 112 is shaped as a diamond having four or six apexes. In some embodiments, the plurality of struts are arranged in a Z-pattern (e.g., each strut is shaped as a “Z”), a zig-zag pattern, a wave pattern, an M-pattern (e.g., each strut is shaped as a “M”), a W-pattern (e.g., each strut is shaped as a “W”), a circular pattern, or any combination thereof. In some embodiments, the plurality of struts 112 are composed of a Nitinol. In some embodiments, the plurality of struts 112 are composed of a metal or a metal alloy.

The stent graft material 104 is coupled to a portion of the tubular body 110, thereby defining an opening 120 in the tubular body 110. For example, in some embodiments, the stent graft material 104 can be sutured or adhered to the stent 102. Though the opening 120 lacks any stent graft material 104, the plurality of struts 112 are present throughout the tubular body 110 and through the opening 120. The stent graft material 104 can be an impermeable fabric (e.g., polyethylene terephthalate (PET) or Dacron®).

The percentage of coverage of the stent 102 by the stent graft material 104 can vary between about 40% and about 60% of the total surface area of the tubular body 110 of the stent 102. For example, as shown in FIG. 1 A, the stent graft materials 104a, 104b, and 104c can cover about 40%, 50%, and 60% of the total surface area of the tubular body 110, respectively. The surface area coverage of the stent graft material 104 and the size of the opening 120 can be selected based on the anatomy of the patient. The stent graft 100 typically has a length that can vary between about 45 millimeters (mm) and 65 mm. The stent graft 100 typically has a diameter that can vary between about 25 mm to about 45 mm. For example, in some embodiments, the diameter of the stent graft 100 can be about 25 mm, about 35 mm, or about 45 mm. The length and diameter of the stent graft 100 can also be chosen by the user (e.g., a medical practitioner) based on the anatomy of the patient. For example, in some embodiments, when using the stent graft 100 for the treatment of an aneurysm, the user can review medical imaging results (e.g., a CT scan) of the patient to assess the exact location of blood vessels branching off of a major blood vessel (e.g., visceral blood vessels branching off of an abdominal or thoracic aorta) and measure the distances between them. Based on this assessment and measurements, the user can then determine which diameter, length, surface area coverage of the stent graft material 104, and size of opening 120 of the stent graft 100 are required to entrap the aneurysm or thrombus while leaving the openings of the branching blood vessels untouched and unobstructed.

Referring specifically to FIG. 1B, the first and second lateral edges 114, 116 of the stent graft 100 are marked superiorly and inferiorly for orientation purposes with a first marker 122. In some embodiments, the first marker is a radiolabel marker. Further orientation is provided by a second marker 124 and a third marker 126 located on opposing locations on the stent 102 and stent graft material 104, respective. When the stent graft 100 is in the cylindrical or tubular configuration, the alignment of the first and second markers 124, 126 form a “target sign” and give a user the perfect antero-posterior view of the stent graft 100. The second marker 124 can be a marking dot that is attached to a strut located in the middle of the uncovered portion of the stent graft 100 (i.e., the opening 120 of the stent graft 100). The third marker 126 can be a circular marker that can be sutured in the center of a posterior aspect of the stent graft material 104.

Still referring to FIGS. 1A and 1B, the stent graft includes multiple rings 118 that are attached to each apex of the struts 112 at the proximal end 106 of the stent graft 100. The rings 118 are located within the inner aspect of the stent graft 100 and are configured to not contact a blood vessel wall when the deployed within a blood vessel. The rings 118 are configured to receive and function as a rail for a string. The apexes of the struts 112 at the distal end 108 of the stent graft 100 are connected together with a monofilament string or suture. The string is configured to be part of the mechanism that reversibly opens and closes the stent graft 100 in a radial direction. For example, tensioning of the string causes the stent graft 100 to be in a radially collapsed configuration while loosening of the string causes the stent graft 100 to be in a radially expanded configuration.

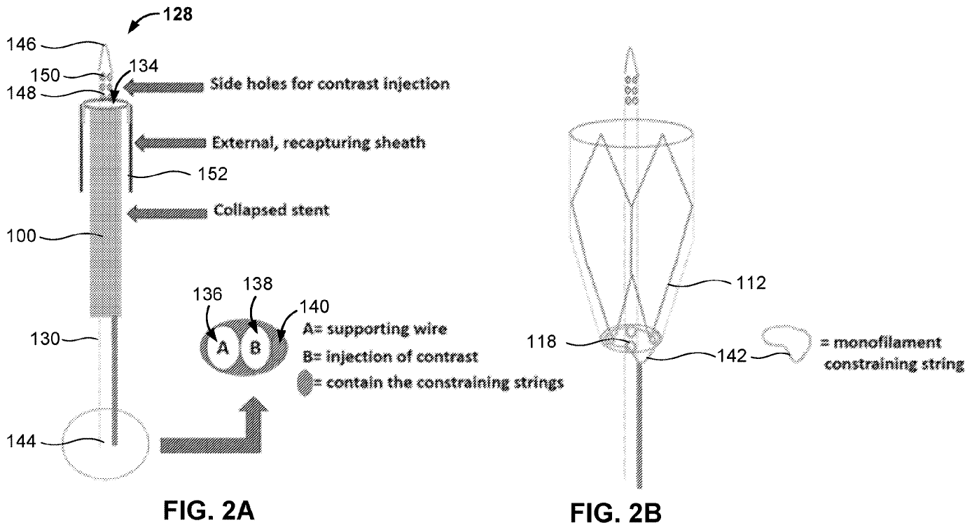

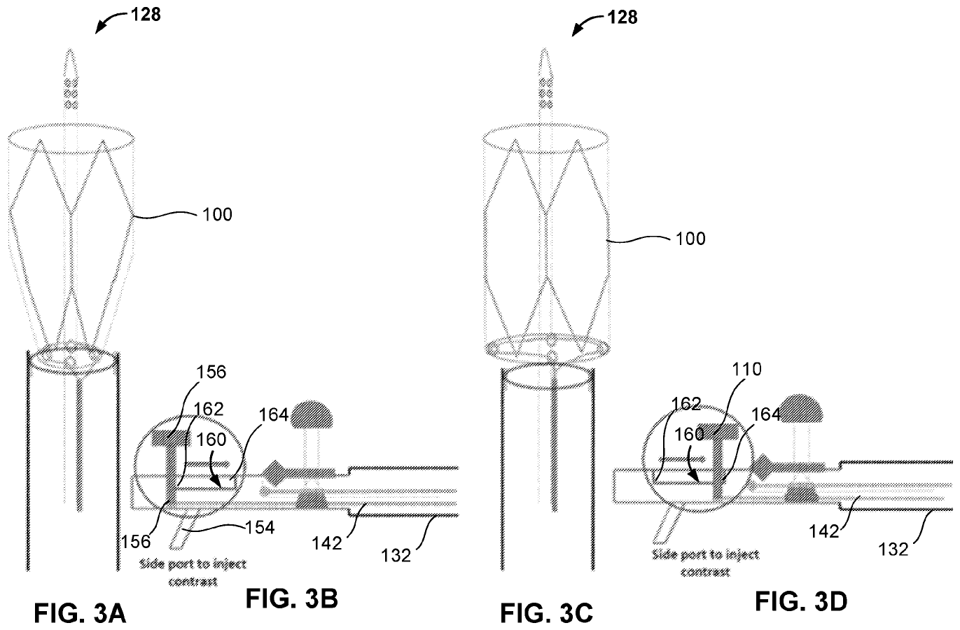

Referring to FIGS. 2A and 2B, the stent graft delivery system 128 includes the stent graft 100, an elongated shaft 130, a sheath 152, and a handle assembly 132 (shown in FIGS. 3A and 3B). The elongated shaft 130 defines a main lumen 134 that further includes three channels, each channel defining a first inner lumen 136, a second inner lumen 138, and a third inner lumen 140. The first inner lumen 136 is configured to receive a supporting wire. The supportive wire is the main wire, which is generally a stiff wire, over which the stent graft is advanced. The second inner lumen 138 is configured to receive a contrast agent fluid, which can be released through the side holes 150 in the distal end 146 of the elongated shaft 130. The third inner lumen 140 is configured to receive the string 142, which is threaded through the rings 118 that are attached to the struts 112, as shown in FIG. 2B. The first and second inner lumens 136, 138 have diameters that are about equivalent in size to each other while the third inner lumen 140 has a diameter that is small than the diameter of the first and second inner lumen 136, 138. The first, second, and third inner lumens 136, 138, 140 have diameters that are less than the diameter of the main lumen 134 such that they are enclosed within the main lumen 134.

The elongated shaft 130 extends between a proximal end 144 and a distal end 146. The distal end 146 has a pointed tip that is configured to ease the insertion of the stent graft delivery system into a blood vessel lumen. The distal end portion 148 of the elongated shaft 130 defines six holes 150 that are located just above the superior edge of the stent graft 100 and in fluid communication with the second inner lumen 138. In some embodiments, the holes 150 are used to inject contrast agent into a blood vessel in order to perform a serial aortogram while adjusting a position of the stent graft 100. The holes 150 advantageously eliminate the need of creating a second arterial access to a blood vessel of the patient solely to perform diagnostic angiograms, which are routinely required in endovascular aortic repair and stent graft deployment. In some embodiments, the elimination of the creation of a second arterial access can be very useful in patients who previously had an arterial access created (e.g., in the groin), an occluded iliac blood vessel on one side, or simply to avoid potential complications related to a second arterial access.

Referring to FIGS. 3A-3D, the stent graft delivery system 128 includes a side port 154 located in the handle assembly 132. The side port 154 in fluid communication with the channel defining the second inner lumen 138 and is configured to receive a contrast agent. Therefore, the contrast agent can be injected through the side port 154. The handle assembly 132 includes a handle 156 (e.g., a T-shaped handle) that is connected to the string 142 at a distal end 158 of the handle 156 and also connected to the handle assembly 132. The handle 156 is configured to slide back and forth within a slot 160 defined by the handle assembly 132. The handle 156 is configured to tension or loosen the string 142 by sliding of the handle 156 to and from a proximal end 162 and a distal end 164 of the slot 160. For example, when the handle 156 is at a proximal end 162 of the slot 160, the string 142 is tensioned, thereby pulling the distal end 106 of the stent graft 100 (e.g., by bringing the struts 112 at the distal end 106 together against the elongated shaft 130) and setting the stent graft 100 in the radially collapsed configuration, as shown in FIG. 3A. When the handle 156 is at a distal end 164 of the slot 160, the string 142 is loosened, thereby loosening the distal end 106 of the stent graft 100 and setting the stent graft 100 in the radially expanded configuration, as shown in FIG. 3C. When the handle 156 is slid at a location halfway between the proximal end 162 and distal end 164 of the slot 160, the string 142 is loosened halfway, thereby setting the stent graft 100 at a 50% radially expanded configuration.

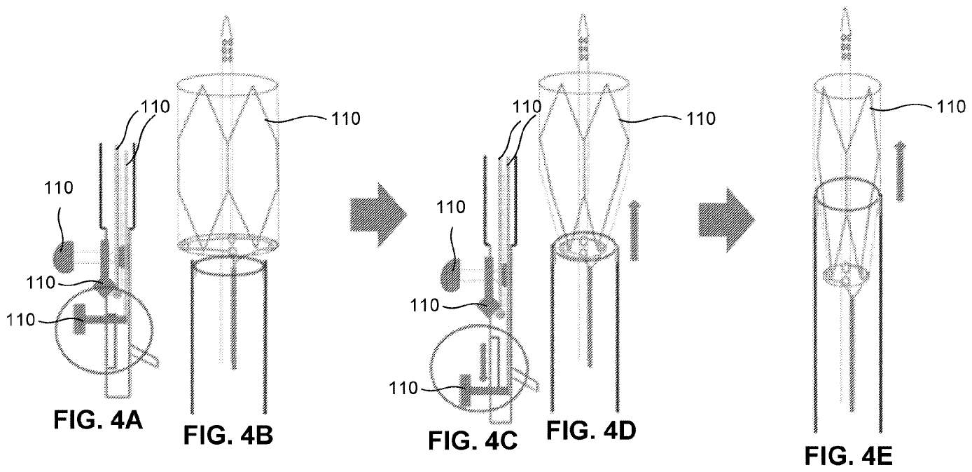

Referring to FIGS. 4A-4E, the sheath 152 is an elongated, tubular sheath that is connected to the handle 156 and configured to be disposed over the stent graft 100 when the stent graft 100 is in the radially collapsed configuration. To advance the sheath 152 towards the proximal end 144 of the elongated shaft 130 and capture the stent graft 100 within the sheath 152, the user advances the sheath 152 in a distal direction, towards the distal end 146, by moving the handle 156 backwards towards a proximal end 162 of the slot 160. Once the stent graft 100 is ready to be expanded and positioned within the blood vessel, the user pulls the sheath 152 in a proximal direction, towards the proximal end 144 of the elongated shaft 130, by moving the handle 156 distally, towards a distal end 164 of the slot 160. If desired, the sheath 152 can be re-positioned over the stent graft 100 after the sheath is pulled in the proximal direction; thus, the sheath 152 can be reversibly positioned over the stent graft 100. By having the struts 112 all connected as one skeleton, the stent graft delivery system 128 is stable, and the user is able to easily recapture the stent graft 100 within the sheath 152.

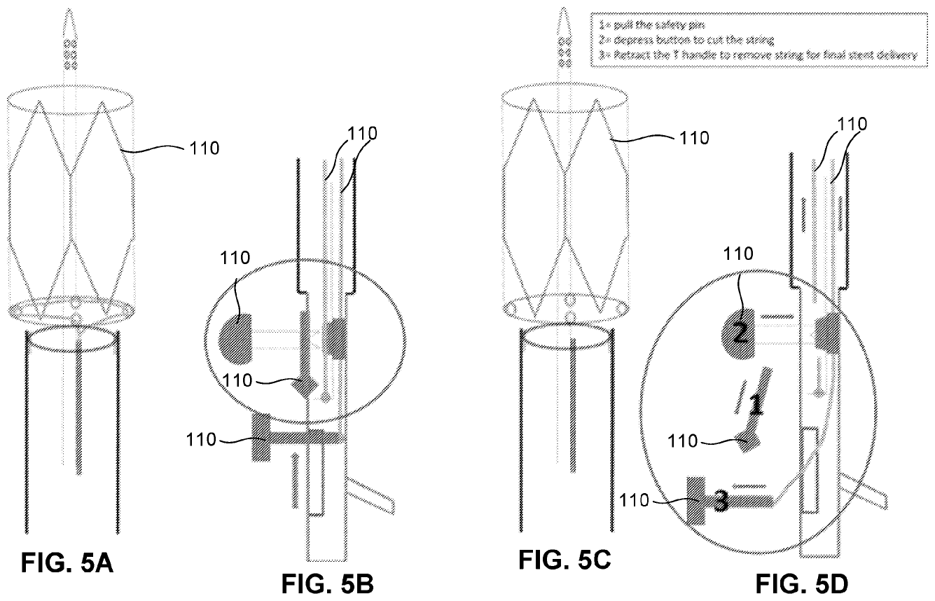

Referring to FIGS. 5A-5D, the handle assembly 132 includes a safety pin 168 and button 172 including a blade 170 disposed at a distal end of the button. The safety pin 168 is configured to prevent the blade 170 from contacting and cutting the string 142. The safety pin 168 is positioned longitudinally, in a direction along an axis defined by the elongated shaft 130 and orthogonally with respect to the button 172. When in use, once the user is satisfied with the position of the stent graft 100 within the blood vessel, the safety pin is removed 168, the string 142 is cut by depressing the button 172, and the handle 156 is retracted, thereby removing the string 142.

A stent graft delivery system including a stent graft may be substantially similar in construction and function in several aspects to the stent graft delivery system 128 and stent graft 100 discussed above, but can include an alternative stent graft and handle assembly design. In some embodiments, the stent graft may have three sets of struts and three separate strings where each string is used to individually control one set of struts. Furthermore, in some embodiments, the stent delivery system may include a handle assembly including three separate handles and buttons to manipulate and cut each string. Such configuration can allow a user to individually and separately manipulate proximal, distal, and middle regions of the stent graft, instead of only being able to manipulate the entire stent graft body, to achieve the desired placement of the stent graft within a blood vessel.

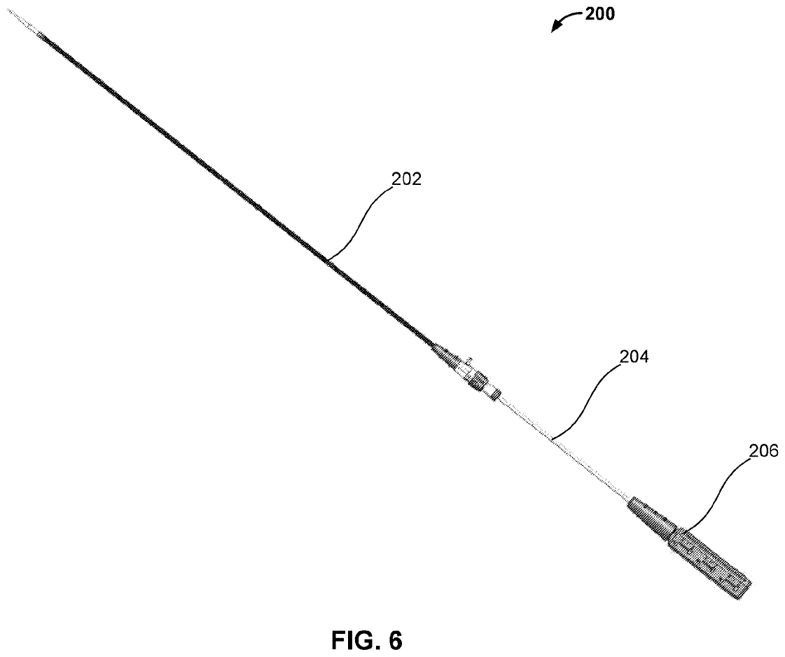

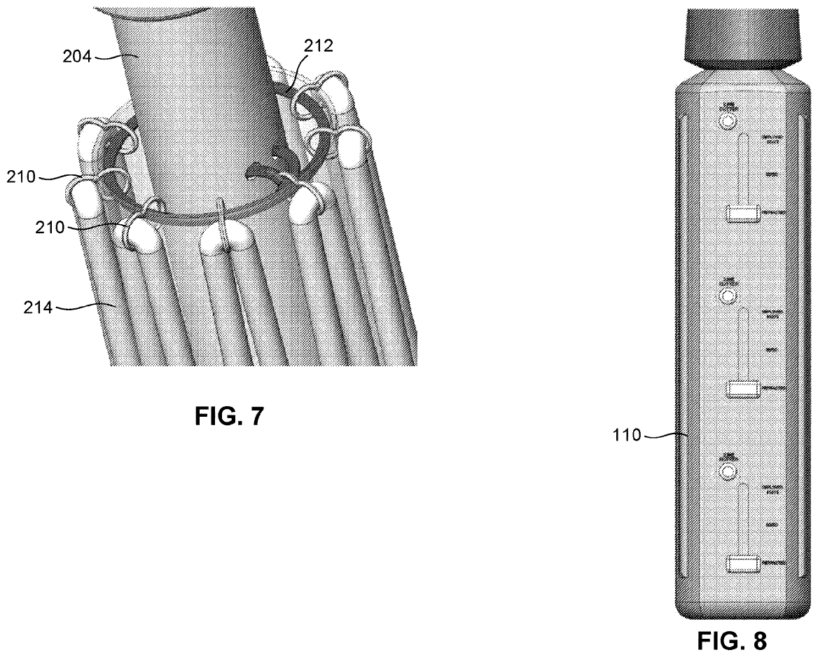

FIGS. 6-10 illustrate an example of a stent graft delivery system 200 including a sheath 202, an elongated shaft 204, a handle assembly 206, and a stent graft 208. Referring specifically to FIG. 7, the stent graft delivery system 200 functions substantially the same as the stent graft delivery system 128 except the design and number of rings 210 is different. For example, the rings 210 have an “8” shape with two connecting, partially open circles, where one circle engages the string 212 and the other circle engages the strut 214.

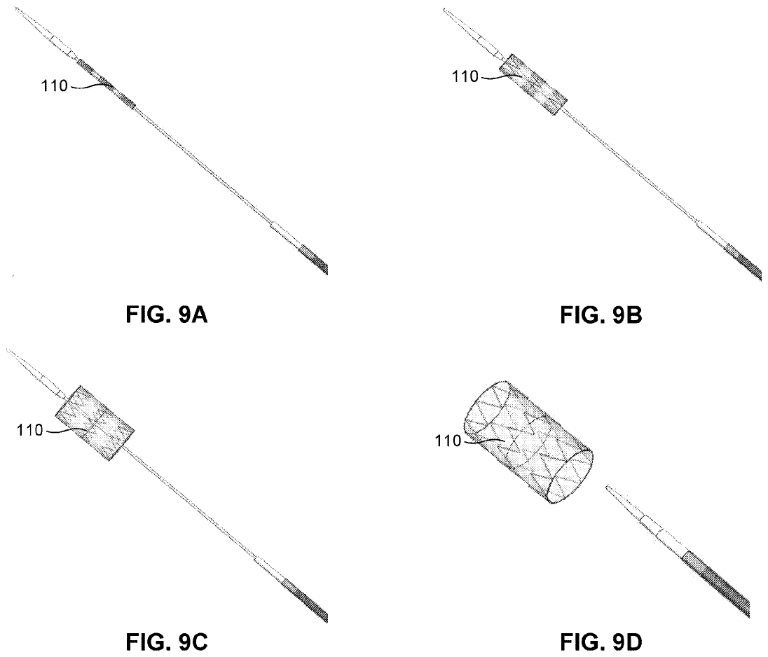

Referring to FIG. 8, another difference between the stent graft delivery systems 128 and 200 is that the handle assembly 206 includes three separate handles and buttons configured to manipulate three strings connected to proximal, middle, and distal regions of the stent graft. The handle assembly 206 includes a first handle 216a, a second handle 216b, and a third handle 216c as well as a first button 218a, a second button 218b, and a third button 218c, which function in the same manner as the handle 156 and the button 172. The first, second, and third handles 216a-c each are configured to slide within a first slot 220a, a second slot 220b, and a third slot 220c, respectively. The first, second, and third slots 220a-c each have three settings where the stent graft 208 is in a radially collapsed configuration, 50% radially expanded configuration, and fully in a radially expanded configuration. The handle assembly 206 includes first, second, and third markers 222a, 222b, and 222c, which each indicate a different stent graft setting. For example, the first marker 222a can read “retracted” to indicate to the user that the stent graft 208 is in a radially collapsed configuration. The second marker 222b can read “50/50” to indicate to the user that the stent graft 208 is in a 50% radially expanded configuration. The second marker 222b can read “deployed state” to indicate to the user that the stent graft 208 is in a fully radially expanded configuration.

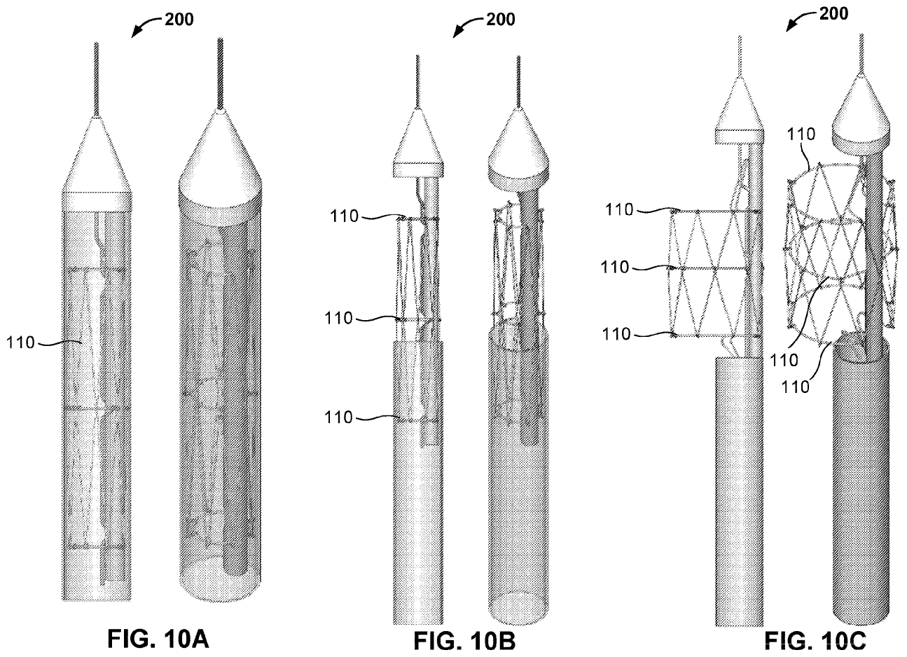

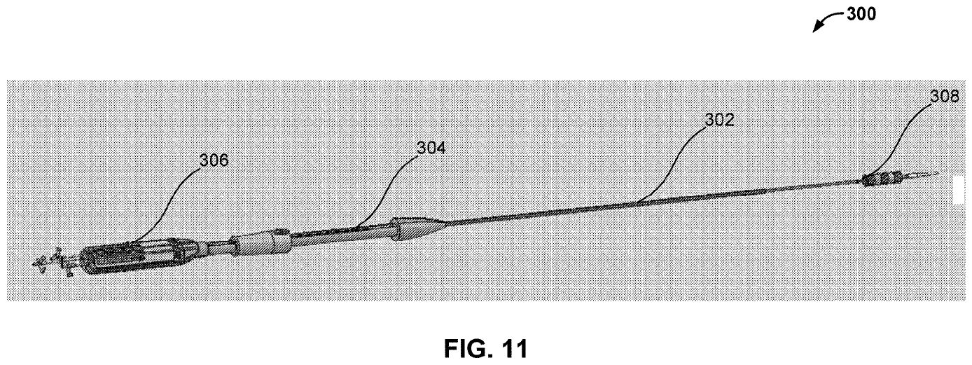

FIGS. 9 and 10 illustrate the stent graft 208, which includes a first plurality of struts 214a, a second plurality of struts 214b, and a third plurality of struts 214c that are individually controlled by a first string 212a, a second string 212b, and a third string 212c, respectively. A distal region of the stent graft 208 is configured to be manipulated (e.g., reversibly transitioned between a radially collapsed configuration and a radially expanded configuration) by the first string 212a that is attached to the first plurality of struts 214a via rings 210. Similarly, a middle region and a proximal region of the stent graft 208 are configured to be manipulated (e.g., reversibly transitioned between a radially collapsed configuration and a radially expanded configuration) by the second string 212b that is attached to the second plurality of struts 214b via rings 210 and by the third string 212c that is attached to the third plurality of struts 214c via rings 210, respectively.

A stent graft delivery system including a stent graft may be substantially similar in construction and function in several aspects to the stent graft delivery system 128 including the stent graft 100 and to stent graft delivery system 200 including the stent graft 208 discussed above, but can include an alternative stent graft and handle assembly design. In some embodiments, the stent graft may have three sets of struts and three separate sets of two strings where each set of two strings is used to individually control one set of struts. Furthermore, in some embodiments, the stent delivery system may include a handle assembly including three separate handles and thumb screws to manipulate, adjust, and/or maintain a position of each strut via the sets of two strings. Such configuration can allow a user to manipulate and maintain a position of the proximal, distal, and middle regions of the stent graft individually and separately, instead of only being able to manipulate the entire stent graft body, to achieve the desired placement of the stent graft within a blood vessel.

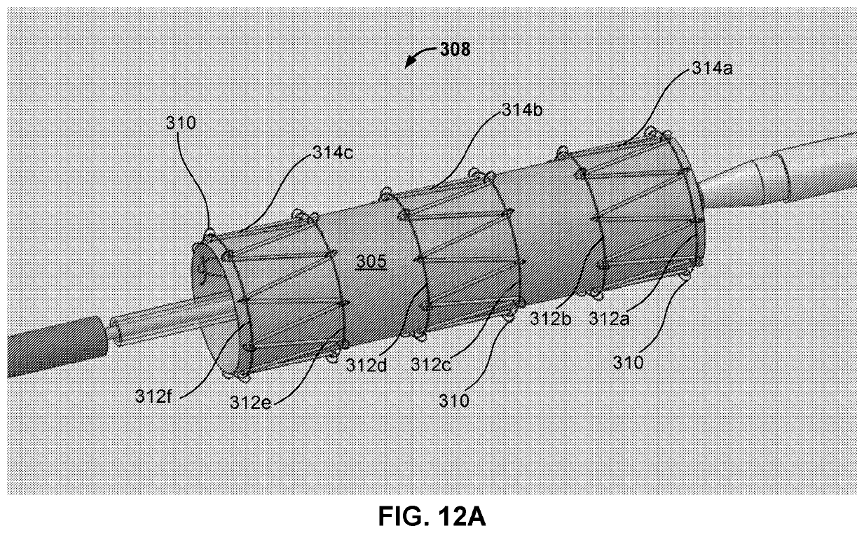

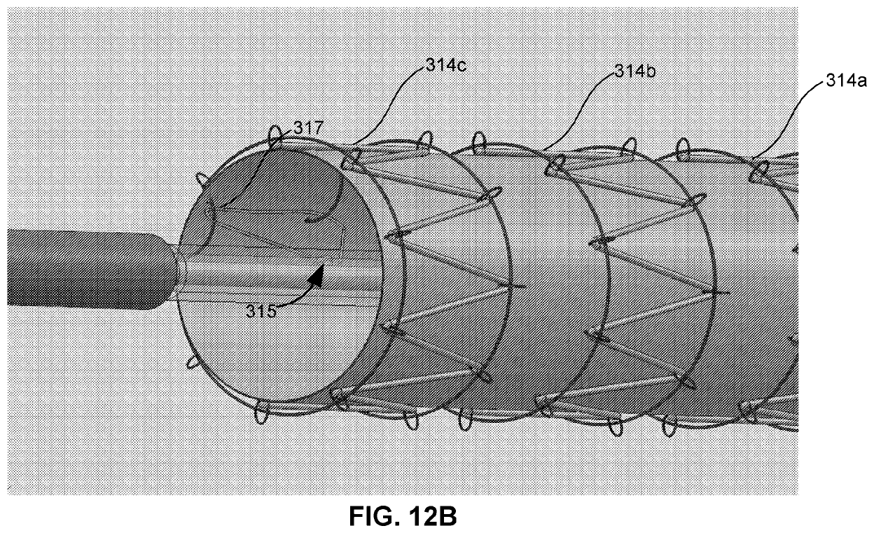

FIGS. 11-16 illustrate an example of a stent graft delivery system 300 including a sheath 302, an elongated shaft 304, a handle assembly 306, and a stent graft 308. Referring specifically to FIG. 11, the stent graft delivery system 300 functions substantially the same as the stent graft delivery systems 128 and 200 except the design and number of strings per strut is different. For example, FIG. 12A illustrates the stent graft 308, which includes a first plurality of struts 314a, a second plurality of struts 314b, and a third plurality of struts 314c having a zig-zag pattern. Each of the first, second, and third plurality of struts 314a, 314b, 314c are individually controlled by a unique set of strings that engage the struts and loop around the circumference of the tubular body of the stent graft 308. The first plurality of struts 314a is individually controlled by a first set of strings including a first string 312a and a second string 312b. The second plurality of struts 314b is individually controlled by a second set of strings including a third string 312c and a fourth string 312d. The third plurality of struts 314c is individually controlled by a third set of strings including a fifth string 312e and a sixth string 312f. Similarly to the stent graft delivery systems described above, the sets of string are configured to be part of the mechanism that reversibly opens and closes the stent graft 300 in a radial direction.

In some embodiments, the first, second, third, fourth, fifth, and sixth strings 312a, 312b, 312c, 312d, 312e, 312f are composed of a suture material. In some embodiments, the suture material is a non-absorbable, sterile surgical suture. In some embodiments, the suture material is a polymeric material. In some embodiments, the polymeric material is polypropylene and/or polyamide. The first, second, third, fourth, fifth, and sixth strings 312a, 312b, 312c, 312d, 312e, 312f are monofilament strings.

A distal region of the stent graft 308 (e.g., the region including the first plurality of struts 314a) is configured to be manipulated (e.g., reversibly transitioned between a radially collapsed configuration and a radially expanded configuration) by the first and second strings 312a, 312b that are attached to the first plurality of struts 314a via rings 310. Similarly, a middle region (e.g., the region including the second plurality of struts 314b) and a proximal region (e.g., the region including the third plurality of struts 314c) of the stent graft 308 are configured to be manipulated (e.g., reversibly transitioned between a radially collapsed configuration and a radially expanded configuration) by the third and fourth strings 312c, 312d that are attached to the second plurality of struts 314b via rings 310 and by the fifth and sixth strings 312e, 312f that are attached to the third plurality of struts 314c via rings 310, respectively.

The rings 310 are attached to the stent material at or near each apex of each strut of the first, second, and third plurality of struts 314a, 314b, 314c. The rings are configured to receive the first, second, third, fourth, fifth, and sixth strings 312a, 312b, 312c, 312d, 312e, 312f and function as a rail for each of the strings that loops around the circumference of the tubular body of the stent graft 308. Thus, the contiguous apexes of each plurality of struts are connected together via the strings that are threaded through the rings 310. Each ring 310 is attached to the stent material 305 via a suture or stitch (e.g., a double stitch). The first, second, and third pluralities of struts 314a, 314b, 314c are also attached to the stent material 304 via stitches. In some embodiments, the rings are composed of the same material (e.g., a suture material) as the first, second, third, fourth, fifth, and sixth strings 312a, 312b, 312c, 312d, 312e, 312f. In some embodiments, the rings can be composed of a different material than the strings.

Referring to FIG. 12B, the elongated shaft 304 defines three holes 315, each hole 315 defined within 180 degrees of each other. A first hole 315 is defined by the elongated shaft 304 in the distal region of the stent graft 308 (e.g., the region including the first plurality of struts 314a), a second hole 315 is defined by the elongated shaft 304 in the middle region of the stent graft 308 (e.g., the region including the second plurality of struts 314b) at 180 degrees from the first hole 315, and third hole 315 is defined by the elongated shaft 304 in the proximal region of the stent graft 308 (e.g., the region including the third plurality of struts 314c) at 180 degrees from the second hole 315. Each of the three holes is configured to receive a wire 317 in the form of a loop that engages the sets of strings of each plurality of struts. Similarly to the stent graft delivery systems described above, the holes 315 are also used to inject a fluid (e.g., a contrast agent, a drug, or the like) into the patient (e.g., into a blood vessel of the patient).

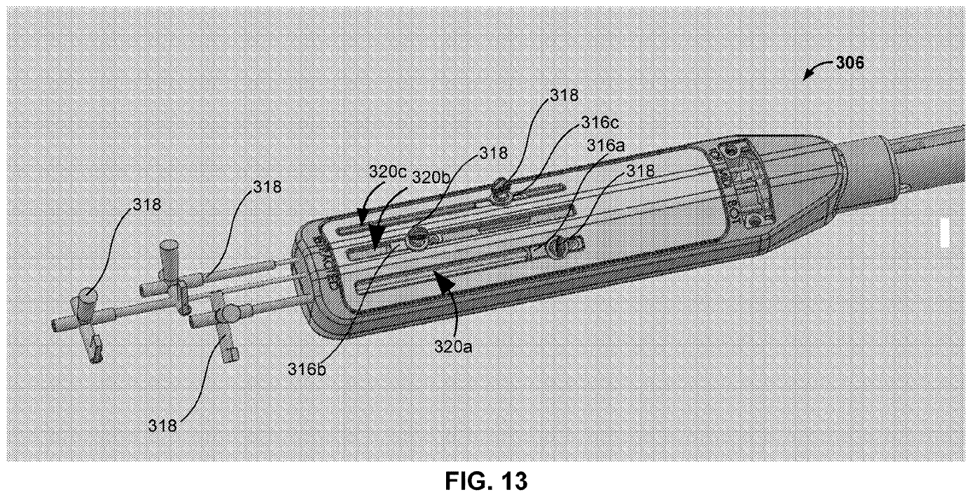

Referring to FIG. 13, the handle assembly 306 includes three separate handles: a first handle 316a, a second handle 316b, and a third handle 316c configured to manipulate the set of two strings connected to the proximal, middle, and distal regions of the stent graft, respectively. Each of the first, second, and third handles 316a, 316b, 316c are configured to enable the proximal, distal, and middle regions of the stent graft 308 to be individually deployed between a radially collapsed configuration to a radially expanded configuration, and all gradual configurations existing therebetween. Each of the first, second, and third handles 316a, 316b, 316c includes a screw 318 (e.g., a thumbscrew). Each of the screws 318 is configured to fix or maintain a position of the proximal, distal, and middle regions of the stent graft individually upon rotation of the screws 318 by a user. In some embodiments, the screws 318 may be used by the user as handles. Similarly to the stent graft delivery systems described above, the first, second, and third handles 316a, 316b, 316c are configured to slide within a first slot 320a, a second slot 320b, and a third slot 320c, respectively. The user is able to control the expansion and contraction of the proximal, distal, and middle regions of the stent graft 308 by sliding the first, second, and third handles 316a, 316b, 316c within the respective first slot 320a, second slot 320b, and third slot 320c. Both ends of each of the three wires 317 that engages each set of two strings are attached to their respective handles. The handle assembly 306 includes first, second, and third valves 324a, 324b, and 324c, which are configured to receive and/or connect to tubing, syringe, and/or a port. Each of the first, second, and third valves 324a, 324b, and 324c is integrally connected to the first, second, and third handles 316a, 316b, 316c, respectively. In some embodiments, the first, second, and third valves 324a, 324b, and 324c are configured to receive a fluid (e.g., a contrast agent, a drug, or the like) that will be delivered into the patient (e.g., into a blood vessel of the patient). The first, second, and third valves 324a, 324b, and 324c are configured to be opened and closed to allow fluid to enter or exit through them.

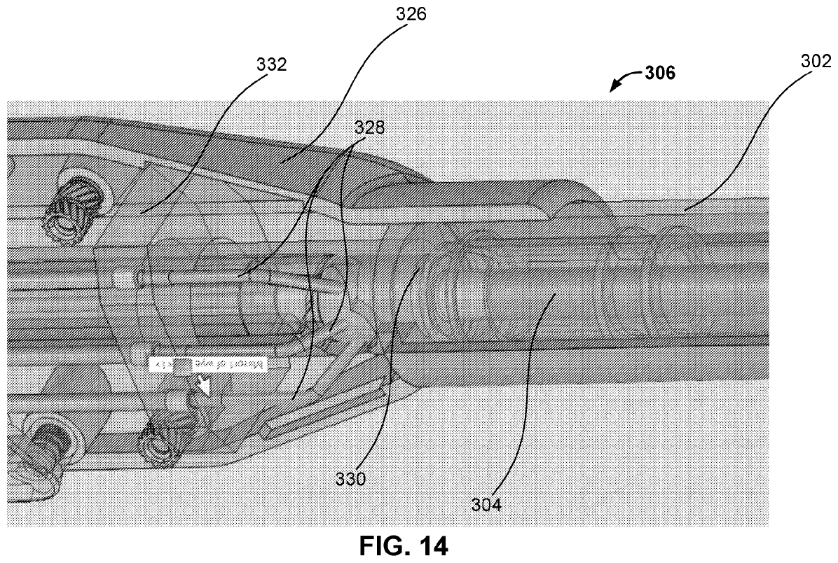

Referring to FIG. 14, the handle assembly 306 includes a housing 326. Within the housing 326, each of the wires that engage the set of two strings is housed separately within the tubes 328. The tubes 328 are fixedly connected to a cylindrical tube 330, which is further in fluid connection with the sheath 302 and elongated shaft. The cylindrical tube 330 has dimensions that are larger than each of the tubes 328. Each of the tubes 328 is further fixedly attached to a retainer 332 that is configured to help stabilize and secure the position of each of the tubes and is configured to seal the tubes. The retainer defines three through-holes, each of which receives a tube 328.

While this specification contains many specific implementation details, these should not be construed as limitations on the scope of any method or device or of what may be claimed, but rather as descriptions of features that may be specific to particular embodiments of particular methods and devices. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described herein as acting in certain combinations and even initially claimed as such, one or more features from a claimed combination can in some cases be excised from the combination, and the claimed combination may be directed to a sub-combination or variation of a sub-combination.

Particular embodiments of the subject matter have been described. Other embodiments are within the scope of the following claims. For example, the actions recited in the claims can be performed in a different order and still achieve desirable results.

What is claimed is:

1. A stent graft comprising:
a stent having a proximal end and a distal end and a tubular body disposed longitudinally therebetween, the stent comprising a plurality of struts forming the tubular body;
a stent graft material coupled to a portion of the tubular body, the stent graft material defining an opening; and
one or more rings engaged with an edge of the stent, the one or more rings configured to receive a string,
wherein tensioning of the string causes the stent graft to be in a radially collapsed configuration.

2. The stent graft of claim 1, wherein the stent graft material is coupled to about 40% to about 60% of the tubular body.

3. The stent graft of claim 1, wherein the stent comprises a first label, and the stent graft material comprises a second label such that the first and second labels are configured to align when the stent graft is in a deployed state.

4. The stent graft of claim 1, wherein the opening defined by the stent graft material is configured to be aligned with a blood vessel opening.

5. The stent graft of claim 1, wherein the tubular body is a metal structure, and wherein the stent graft material is an impermeable fabric.

6. The stent graft of claim 1, wherein loosening of the string causes the stent graft to be in a radially expanded configuration.

7. The stent graft of claim 1, wherein the plurality of struts are interconnected.

8. The stent graft of claim 1, wherein the plurality of struts are arranged in a diamond-shaped pattern.

9. The stent graft of claim 8, wherein the plurality of struts is a first plurality of struts, and the stent further comprises a second plurality of struts and a third plurality of struts arranged in a diamond-shaped pattern, and wherein the stent graft further comprises one or more rings engaged at one or more apexes of one or more diamonds forming the diamond-shaped pattern.

10. A stent graft delivery system comprising:
an elongated shaft having a proximal end and a distal end;
a stent graft having a tubular body disposed over the elongated shaft, the stent graft comprising a plurality of struts forming the tubular body and one or more rings engaged with an edge of the stent, the one or more rings configured to receive a string; and
a handle assembly extending from a proximal end of the elongated shaft, the handle assembly attached to the string and configured to tension or loosen the string,
wherein the stent graft is configured to be in a radially expandable configuration when the string is loosened, and
wherein the stent graft is configured to be in a radially collapsed configuration when the string is tensioned.

11. The stent graft delivery system of claim 10, wherein the stent graft is configured to reversibly transition between a radially expandable configuration and the radially collapsed configuration.

12. The stent graft delivery system of claim 10, wherein the handle assembly further comprises a blade configured to sever the string.

13. The stent graft delivery system of claim 12, wherein the stent graft is configured to be deployed and engaged with a wall of a blood vessel after the string is severed.

14. The stent graft delivery system of claim 12, wherein the handle assembly further comprises a safety pin configured to prevent the blade from contacting the string.

15. The stent graft delivery system of claim 10, further comprising an elongated, tubular sheath configured to be disposed over the stent graft when the stent graft is in the radially collapsed configuration.

16. The stent graft delivery system of claim 10, wherein the elongated shaft is hollow and defines a lumen, and wherein the handle assembly further comprises a side port in fluid communication with the lumen of the elongated shaft.

17. The stent graft delivery system of claim 10, wherein the plurality of struts are reversibly collapsible.

18. The stent graft delivery system of claim 10, wherein the plurality of struts are arranged in a diamond-shaped pattern.

19. The stent graft delivery system of claim 18, wherein the plurality of struts is a first plurality of struts, and the stent graft further comprises a second plurality of struts and a third plurality of struts arranged in a diamond-shaped pattern, and wherein the stent graft further comprises one or more rings engaged at one or more apexes of one or more diamonds forming the diamond-shaped pattern.

20. The stent graft delivery system of claim 19, wherein the string is a first string, wherein the one or more rings of the second plurality of struts is configured to receive a second string, wherein the one or more rings of the third plurality of struts is configured to receive a third string, and wherein the handle assembly is attached to the second and third strings.
